(19) United States
(12) Patent Application Publication
(10) Pub. No.: US 2022/0257545 A1
(43) Pub. Date: Aug. 18, 2022

(54) pH STABLE UREA COMPOSITION AND USE THEREOF

(71) Applicant: Orenova Development Company LLC, Mamaroneck, NY (US)

(72) Inventors: Barrie Levitt, Mamaroneck, NY (US); Derek Ganes, Toronto (CA); Avraham Yacobi, Englewood, NJ (US)

(21) Appl. No.: 17/666,197

(22) Filed: Feb. 7, 2022

Related U.S. Application Data
(60) Provisional application No. 63/149,027, filed on Feb. 12, 2021.

(51) Int. Cl. A61K 31/17; A61K 31/191

(52) U.S. Cl. CPC A61K 31/17; A61K 31/191

(57) ABSTRACT

The invention includes a composition for the treatment of a skin disorder, in particular topical treatment. Embodiment of the composition include at least 20% w/w urea, and a pH stabilizing component, where the pH stabilizing component includes a hydroxy acid such as lactic acid and a polyhydroxy acid or lactone thereof, such as gluconolactone. The composition has an initial pH of less than 6.2 and the composition has a pH less than about 6.2 after storage for at least four weeks. The invention is also a method of treating a skin or nail disorder by topically administering the composition. The invention is also a method of improving pH stability of a urea formulation that has greater than about 20% w/w urea.

The present application claims priority to U.S. Provisional Patent Application No. 63/149,027, filed Feb. 12, 2021, the entire contents of which are incorporated herein by reference.

### FIELD OF THE INVENTION

The present invention relates generally to topical urea compositions. More particularly the invention is a topical urea composition having a high urea concentration that exhibits pH stability over an extended time and uses of the composition for the treatment of skin disorders.

### BACKGROUND

Urea is a normal component of human blood and has been used orally and intravenously in clinical settings for the treatment of a wide range of disease states. Urea is generally recognized as safe in these uses and is safe for topical therapy as well. In fact, “There is no evidence in the available information on urea that demonstrates or suggests reasonable grounds to suspect, a hazard to the public when it (urea) is used at levels that are now current or that might reasonably be expected in the future.” (Select Committee on GRAS Substances Opinion: Urea, Type of Conclusion: 1 ID Code: 57-13-6, 1976, 21 CFR Section: 184.1923, U.S. Food and Drug Administration Website., see also <www.epa.gov/pesticides/chem_search/cleared_reviews/csr_PC-085702_19-Mar-02_001.pdf>.)

Urea has been administered intravenously in neurology and neurosurgery applications, for example as an osmotic diuretic to reduce intracranial pressure, to mitigate cerebral edema and to reduce brain volume, especially in the surgical setting. Urea has also been used systemically in the prophylaxis and therapy of sickle cell crisis, in the reduction of intraocular pressure in glaucoma, and in the management of the syndrome of inappropriate anti-diuretic hormone secretion.

Topical urea has widespread use in dermatologic practice and has a long history of clinical use in the treatment of skin diseases. Since 1900, urea has been employed as a proteolytic agent for wound debridement, and as a topical bacteriostatic agent in wounds. Since the 1940's, urea has been used as a moisturizer, and since the 1950s, as a keratolytic agent. The use of urea is generally accepted by the dermatological, podiatric, and general medical community.

While there are no “FDA” approved uses of urea as an active ingredient in a topical product, among its unapproved uses, urea products have been marketed for:
Debridement and promotion of normal healing of hyperkeratotic surface lesions, particularly where healing is retarded by local infection, necrotic tissue, fibrinous or purulent debris or eschar.
Hyperkeratotic conditions such as dry, rough skin, dermatitis, psoriasis, xerosis, ichthyosis, eczema, keratosis, keratosis pilaris, keratosis palmaris, keratoderma, corns, calluses
Damaged, ingrown and devitalized nails.

In addition, as discussed below, urea has been successfully used topically, orally, ophthalmically, intra-amniotically, and intravenously in a variety of clinical conditions.

Scores of clinical studies have been published in a broad spectrum of disease entities including: ichthyosis, xerosis, atopic dermatitis/eczema, psoriasis, seborrheic dermatitis, emollient/keratolytic, onychomycosis, radiation-induced dermatitis, chemical nail avulsion/dystrophic nails, tinea pedis, penetration enhancement, antipruritic, gingival disease, hand-foot syndrome, malignancies, sickle cell anemia, wound debridement, antimicrobial, increased intracranial pressure/cerebral edema, acute glaucoma, syndrome of inappropriate antidiuretic hormone secretion, and as an abortifacient.

Urea has been used in topical therapy for treating: xerosis, psoriasis, seborrheic dermatitis, ichthyosis vulgaris, eczema, onychomycosis, tinea pedis, pruritus, dystrophic nails, radiation dermatitis, hand-foot syndrome, malignancies (basal cell and squamous cell carcinomas as well as melanoma and uterine cervical cancer), wound debridement, and as an antimicrobial agent. Topical exposure for some disorders (e.g., psoriasis, seborrheic dermatitis, eczema, onychomycosis, tinea pedis) is often accompanied by a co-active ingredient whose penetration into the skin is intended to enhance efficacy.

Urea has been used in systemic oral therapy for treating a wide range of disorders including, for example: hepatic primary and metastatic neoplasms, diuretic, gingival disease, sickle cell anemia, intracranial hypertension, and syndrome of inappropriate antidiuretic hormone secretion (SIADH). It has also been used in ophthalmic therapy for treating skin cancer, near the eye (basal cell, squamous cell and melanoma)—topically, and acute glaucoma—intravenously. It has found use in intra-amniotic therapy as an abortifacient. Intravenously administered urea has been used for treating intracranial hypertension and cerebral edema, sickle cell crisis, treatment and prophylaxis, syndrome of inappropriate antidiuretic hormone secretion (SIADH), and acute glaucoma.

### SUMMARY OF THE INVENTION

While the studies herein above generally related to the effectiveness of urea, none identify any issues related to the formulation used in the studies. For example, there are no studies addressing whether the formulations remain stable and efficacious upon storage. As described further herein, it has been identified that urea formulations, particularly those containing high concentrations (≥20%) of urea, may lose efficacy over time. It was further identified that the loss of efficacy correlates to an increase in pH of the formulation. There is a continuing need to develop safe and effective urea formulations that remain stable and effective over time.

The invention includes a composition for the treatment of a skin disorder, in particular a topical treatment. Embodiments of the composition include at least 20% w/w urea, and a pH stabilizing component, where the pH stabilizing component includes a hydroxy acid and a polyhydroxy acid or lactone thereof; the composition has an initial pH of less than 6.2, and the composition has a pH less than about 6.2 after storage for at least four weeks. In some embodiments, the composition has an initial pH of less than 6.0, and the composition has a pH less than about 6.0 after storage for at least four weeks. In embodiments, the hydroxy acid is present in an amount of from about 0.25% to about 15%, for example from about 0.25% to about 2%. In embodiments, the polyhydroxy acid or lactone thereof is present in an amount of from about 0.5% to about 8%. In embodiments, the hydroxy acid is an α-hydroxy acid, for example lactic acid. In embodiments, the polyhydroxy acid is gluconic acid and can be in the form of gluconolactone. In particular embodiments, the hydroxy acid is lactic acid and the polyhydroxy acid or lactone thereof is gluconolactone.

In embodiments, the pH of the composition is less than about 6.2 or less than 6.0 after 4 weeks of storage at 25° C. and 60% Relative Humidity (RH). In embodiments, the initial pH is from about 4.8 to about 6.2. In embodiments, the initial pH is from about 5.0 to about 6.0. In embodiments, the pH is from about 4.8 to about 6.2 after storage for 4 weeks at 25° C. and 60% RH. In embodiments, the pH is from about 5.0 to about 6.0 after storage for 4 weeks at 25° C. and 60% RH. In embodiments, the pH of the composition remains less than about 6.2 or less than about 6.0 after storage for about 8 weeks.

In embodiments, the composition includes from about 20% to about 40% w/w urea, for example, about 40% w/w urea. Embodiments having about 20% to about 40% w/w urea can include about 0.25% to about 2% lactic acid as the hydroxy acid, for example, about 1% lactic acid. Embodiments having about 20% to about 40% w/w urea can include about 0.5% to about 8% gluconolactone as the polyhydroxy acid or lactone thereof, for example about 2% gluconolactone. Particular embodiments having about 20% to about 40% w/w urea can include about 1% w/w lactic acid as the hydroxy acid and about 2% w/w gluconolactone as the polyhydroxy acid or lactone thereof. Embodiments of compositions of the invention can include about 40% urea, about 1% w/w lactic acid and about 2% w/w gluconolactone.

Embodiments of the invention further include a method of treating a skin or nail disorder by topically administering to a subject having a skin or nail disorder with said disorder an effective amount of a composition according to any embodiment of the invention. In embodiments, the skin disorder or nail disorder is a hyperkeratotic condition, for example, dry and rough skin, dermatitis, psoriasis (including plaque psoriasis), verruca vulgaris (warts), lichen simplex chronicus (LSC) xerosis, ichthyosis, eczema, keratosis, keratosis pilaris, keratosis palmaris, keratoderma, corns, calluses lichen planus, pityriasis rubra pilaris, dermatophyte infection and any skin condition associated with scales. In embodiments, the skin disorder is ichthyosis or psoriasis. In embodiments, the skin or nail disorder is one of onychomycosis, psoriatic nails, damaged nails, ingrown nails or devitalized nails.

Embodiments of the invention further include the use of a composition according to any embodiment of the invention in preparation of a medicament for treatment of a skin disorder characterized by hyperkeratosis.

Embodiments of the invention further include a method of improving pH stability of a urea formulation that has greater than about 20% w/w urea, by adding a stabilizing amount of a combination of a hydroxy acid and a polyhydroxy acid or lactone thereof. In embodiments, the formulation has an initial pH of less than about 6.2 and, after storage for four weeks, the pH is of less than about 6.2. In embodiments, the formulation has an initial pH of less than about 6.0 and, after storage for four weeks, the pH is of less than about 6.0. In embodiments, the formulation contains from about 20% w/w to about 40% w/w urea.

Further objectives and advantages, as well as the structure and function of preferred embodiments will become apparent from a consideration of the description, and examples.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

In the description and examples that follow, all temperatures are set forth in uncorrected degrees Celsius. Unless otherwise indicated, all parts and percentages are by weight. As used herein, the term “about” refers to plus or minus 10% of the indicated value. Unless otherwise stated or made clear by context, weight percentages are provided based on the total amount of the composition in which they are described. As used herein, the singular forms “a,” “an,” and “the” include plural referents unless the context clearly dictates otherwise.

As used herein, the terms “treatment” and “treating” of a skin disorder, and equivalent expressions thereof, mean to at least improve the appearance by reducing cornification or improving a physical characteristic of the skin or nails. Physical characteristics include texture (smoothness or roughness), color or redness, presence of sores, itching, and scaling. In some cases, treating includes healing or elimination of the disorder.

Urea is an organic compound that has been used clinically for dermatological diseases for more than a century. Urea is a potent emollient and keratolytic agent, making urea an effective monotherapy for conditions associated with dry and scaly skin. While the technical literature abounds with examples of uses of urea, there are few, if any, rigorous, adequate or well controlled, double blind studies comparing urea to its vehicle for the treatment of skin diseases and disorders meeting current clinical standards. In particular, there do not appear to be any studies documenting the pH characteristics of urea formulations, and the impact of formulation pH on efficacy.

Ichthyosis: The most common form of ichthyosis is ichthyosis vulgaris; however, a variety of other disorders present with ichthyosiform scale. Urea has been shown to be useful as a therapy in many of these disorders. A number of studies and expert opinions have supported the use of topical urea for these indications. There are reports of occasional mild burning or irritation associated with the use of some topical urea preparations. In most cases, topical treatments of urea use relatively low concentrations of less than about 20%, and most frequently 10% or less. Krug et al. suggested the use of 5-12% urea and 5-10% lactic acid in newborns with congenital ichthyosis together with regular bathing. (Krug M, Oji V, Traupe H, Berneburg M. Ichthyoses—Part 2: Congenital ichthyoses. J Dtsch Dermatol Ges. 2009 7(7):577-588.)

Xerosis: The use of urea in xerosis typically involves lower concentrations of urea, often 10%, although a few studies have used more than 20%, for example 40% urea. (See, e.g., Ademola J, Frazier C, Kim S J, Theaux C, Saudez X. Clinical Evaluation of 40 Urea and 12 Ammonium Lactate in the Treatment of Xerosis. American Journal of Clinical Dermatology. 2002 3(3):217-222.)

Atopic Dermatitis/Eczema: The use of urea in atopic dermatitis has been studied most often using a concentration of 10% and frequently in combination with 1% hydrocortisone. In some cases, a 20% concentration of urea has been used (Roth, HL. Atopic Dermatitis: treatment with a urea-corticosteroid cream. Cutis. 1973 11:237-239.) Nearly all studies demonstrated some benefit. Some studies reported occasional stinging and burning, which the authors attributed to urea. Gip and Karltorp conducted a double-blind comparison between Diproderm (0.05% betamethasone-17,21-dipropionate) ointment and Calmuril-HC (1% hydrocortisone in cream base of 10% urea and 1% lactic acid). (Gip L, Karltorp N. The rapidity of the effect of different types of topical corticosteroids: a double-blind comparison between Diproderm ointment 0.05 percent and Calmuril-Hydrocortisone 1 percent. Curr Ther Res Clin Exp. 1974 16(4):300-305.) The authors observed that Diproderm was more effective than Calmuril-HC. One subject worsened on Calmuril-HC. Nine subjects in the Calmuril-HC group noted smarting.

Psoriasis/Seborrheic Dermatitis: The majority of studies of urea in psoriasis were performed as part of combination therapies with either dithranol or bifonazole. Urea as a 10-12% cream alone was effective and had few side effects. The side effects reported were occasional stinging and burning. Shemer et al. conducted an open-label study of patients with scalp psoriasis or scalp seborrheic dermatitis treated with an ointment of 40% urea and 1% bifonazole. (Shemer A, et al. Treatment of scalp seborrheic dermatitis and psoriasis with an ointment of 40% urea and 1% bifonazole. Int. J. Dermatol. 2000 39(7):532-534.)

Emollient/Keratolytic Effects: Various in vitro and in vivo studies have established the emollient and keratolytic properties of urea in the clinical setting. Common study endpoints include transepidermal water loss reduction, stratum corneum hydration, as assessed by surrogate measures, as well as clinical assessment. Many reports do not identify the concentrations of Urea that are used, although where reported, most others use lotions, ointments and creams with 10% or less urea. Banerjee et al. studied the emollient and keratolytic properties of urea, including a 20% urea lotion. (Banerjee P K, et al. Topical urea in dermatology. Indian J Dermatol. 1990 35(1):17-24.) Gloor et al. studied the clinical effect of salicylic acid and 40% urea applied according to the standardized New German Formulary and found that 40% urea was keratolytic using the silver nitrate test (stratum corneum blackened with silver nitrate and a photographic developer, with a chromameter used to determine the amount of discoloration produced by the study products) (Gloor M et al. Clinical effect of salicylic acid and high dose urea applied according to the standardized New German Formulary. Pharmazie. 2001 56(10):810-814).

Onychomycosis: Urea has been used in combination with a variety of antifungal agents to achieve partial cures of onychomycosis. In most cases, relatively high concentrations (40%) were used. (See, Shemer A et al. Treatment of onychomycosis using 40% urea with 1% bifonazole. Harefuah. 1992 122(3):159-160; Tsuboi R et al. Topical treatment of onychomycosis by occlusive dressing using bifonazole cream containing 40% urea. Nihon Ishinkin Gakkai Zasshi. 1998 39(1):11-16; Baran R, Tosti A. Chemical avulsion with urea nail lacquer. Journal of Dermatological Treatment. 2002 13(4):161-164 (40% urea); Baran R, Coquard F. Combination of fluconazole and urea in a nail lacquer for treating onychomycosis. Journal of Dermatological Treatment. 2005 16(1):52-55 (1% fluconazole and 20% urea); Bassiri-Jahromi S et al. A comparative evaluation of combination therapy of fluconazole 1% and urea 40% compared with fluconazole 1% alone in a nail lacquer for treatment of onychomycosis: therapeutic trial. J. Dermatological Treatment. 2011:1-4.)

Chemical Nail Avulsion/Dystrophic Nails: Compositions containing urea at 20-40% concentration have been used successfully under occlusion to achieve chemical avulsion of the nail in a number of studies involving many patients. Side effects of maceration and irritation have been reported. (See, Arievich et al. Certain new principles and methods in the treatment of patients with onychomycoses. Vestn Dermatol Venerol. 1960 34:30-35 Farber E M, South D A. Urea ointment in the nonsurgical avulsion of nail dystrophies. Cutis. 1978 22(6):689-692; Port M, Sanicola K F. Nonsurgical removal of dystrophic nails utilizing urea ointment occlusion. J Am Podiatry Assoc. 1980 70(10):521-523; Buselmeier T J. Combination urea and salicyclic acid ointment nail avulsion in nondystrophic nails: a follow-up observation. Cutis. 1980 25(4):397, 405; South D A, Farber E M. Urea ointment in the nonsurgical avulsion of nail dystrophies—a reappraisal. Cutis. 1980 25(6):609-612; Pinner T A, et al. Study of efficacy of urea compound versus emollient cream in avulsive therapy of dystrophic nails. Cutis. 1990 46(2):153, 156-157.)

Tinea Pedis: While urea monotherapy has been reported to have antimicrobial properties, it has been studied in combination with antifungal creams and appears to enhance efficacy over topical antifungal monotherapy with only rare instances of irritation, which is self-limited. In most cases, concentrations of 10-20% are used. Use of a 40% urea cream in the treatment of moccasin tinea pedis has been reported. (Elewski B E, et al. The use of 40% urea cream in the treatment of moccasin tinea pedis. Cutis. 2004 73(5):355.) Patients were treated using 40% urea cream daily and ciclopirox cream twice daily for two to three weeks and 100% cure was observed in all patients, although one patient complained of irritation in the toe webs.

Malignancies: Urea has been used orally in high doses as well as instilled locally into cervical and stomal areas with little negative systemic effect. Urea has been applied in powder form as well as in solution and ointment form topically without a significant negative effect. Urea solution has also been safely applied to the eyes. For topical use, concentrations under 20% are typically used

Other uses: A number of studies support the capacity of urea to enhance penetration of drug substances into the skin. A variety of substances have been studied, including topical steroids and topical antifungal drugs. Urea solutions have also been reported as antipruritics. (See Swanbeck G, Rajka G. Antipruritic effect of urea solutions. An experimental and clinical study. Acta Derm. Venereol. 1970 50(3):225-227.) Oral and intravenous urea therapy has been used for the treatment of Sickle Cell Anemia, and there have been some studies of the antibacterial effects of urea.

In developing a new urea formulation for topical use, an initial composition of urea cream was tested for the treatment of cornification disorders to establish its safety and efficacy relative to placebo/vehicle in controlled double-blind studies. Use of urea for the treatment of cornification disorders generally requires relatively high concentrations of urea, typically about 20% or higher, and usually about 40% urea. This is in contrast to many topical urea formulations which generally use lower concentrations of urea, typically about 10% or less, although in some cases urea concentrations as high as about 20% are used.

The initially prepared urea formulation used typical ingredients present in existing urea formulations. The formulation included 40% Urea, 0.25% Benzoic Acid, 1.50% Sodium Lactate Solution (50%), 0.50% Carbopol 980, 2% White Petrolatum, 4% Mineral Oil, 4% Emulsifying Wax, 0.10% Butylated Hydroxytoluene, 0.10% Edetate Disodium, 6.65% Propylene Glycol, 0.21% NaOH, and 40.69% Purified Water. A double-blind, parallel group study was conducted to evaluate this urea cream for the treatment of ichthyosis in adult subjects for one-month. A single center, randomized, double-blind, parallel group, comparison design, single concentration study was conducted on 19 adult human male and non-pregnant female patients affected with ichthyosis. (Details are given in the Examples presented below.) To assess efficacy, the test formulation was compared to vehicle, i.e. the identical formulation prepared without urea. It was surprisingly found that there was no statistically significant difference in the clinical outcome when the urea containing composition was compared to vehicle alone.

To better characterize the test results from the clinical study, the pH of the urea formulation was evaluated. It was unexpectedly observed that the pH of the urea containing formulation rose significantly from the time it was initially manufactured to the time tested. At the time of manufacture, the pH of the urea formulation was 5.43. After manufacture, the formulation was packaged into tubes and stored under normal conditions of 25° C. and about 60% Relative Humidity (RH). Following packaging and testing in clinical trials, i.e. about three months after manufacture, it was found that the pH had risen to about 9.42. These observations, and the results after storage under different temperature and RH conditions is summarized in Table 1.

It was hypothesized that the lack of efficacy of the urea formulation as compared to vehicle was due to this pH change. The pH change may have caused some decomposition of the urea during storage, resulted in decomposition of urea upon storage or application, or may have changed the nature of the vehicle so as to reduce effectiveness of the urea. However, the urea content after storage at 12 months at 25° C. and 60% RH was 100.7%, indicating that decomposition of the urea was unlikely, and that the decreased efficacy was more likely related to the pH of the cream.

TABLE 1
Influence of Temperature and Relative Humidity on pH

| Temperature | Relative | Baseline | | | |
|---|---|---|---|---|---|
| (° C.) | Humidity | (Initial) | 1 month | 2 months | 3 months |
| 25 | 60% | 5.43 | 9.31 | 9.41 | 9.42 |
| 30 | 65% | 5.43 | 9.35 | | 9.45 |
| 40 | 75% | 5.43 | 9.51 | 9.70 | 9.67 |

Attempts were then made to manufacture a pH stable formulation. A number of formulations were prepared using varying amounts of acidic and salt components in attempts to stabilize the pH of the composition and maintain efficacy. The components used and relative amounts are shown in Table 2 below. The “Initial” Comparative Composition corresponds to the composition initially prepared and clinically tested above. Experimental details are given in the Examples that follow. Table 3 shows the initial pH of the formulation and the pH after storage at 25° C. and 60% RH after 8 weeks. All composition included 40% w/w urea, 2% white petrolatum, 4% Mineral Oil, 4% Emulsifying wax, 0.1% butylated hydroxytoluene (BHT), 0.5% Carbomer (Carbopol 980), 0.1% edetate disodium (Disodium EDTA), and 6.65% propylene glycol. Water was present in a range from about 38 to about 41%.

TABLE 2
Components in Comparative Examples

| Component | COMPARATIVE COMPOSITION | | | | | | |
|---|---|---|---|---|---|---|---|
| (wt %) | Initial | A | B | C | D | E | F |
| Benzoic acid | 0.25 | 0.25 | 0.25 | 0.25 | * | 0.25 | 0.25 |
| Sodium Lactate | 1.5 | | | | | 0.75 | |
| Solution (50%) | | | | | | | |
| NaOH (18% | 1.16** | 1.15 | | 1.15 | 1.15 | 1.15 | |
| solution)** | | | | | | | |
| Tromethamine | | | 1.65 | | | | 1.65 |
| (Tris), 40% | | | | | | | |
| Citric Acid | | | | 0.463 | | | 0.463 |
| (Anhydrous) | | | | | | | |
| Sodium | | | | 2.233 | | | 2.233 |
| Citrate | | | | | | | |

* In Comparative composition D, 0.25% benzoic acid was replaced with 0.25% benzyl alcohol
**The Initial Comparative composition was prepared with 0.21% NaOH, which is equivalent to about 1.16% of an 18% NaOH solution.

TABLE 3
pH Changes in Comparative Examples

| Comparative | | pH at 8 weeks | |
|---|---|---|---|
| Example | Initial pH | (25° C. storage) | Change in pH |
| A | 6.8 | 7.9* | 1.1 |
| B | 6.46 | 7.62 | 1.16 |
| C | 6.47 | 7.59 | 1.12 |
| D | 7.29 | 8.44* | 1.15 |
| E | 7.11 | ** | ** |
| F | 5.5 | 7.11 | 1.61 |

*Recorded at 7.5 weeks
** Testing terminated at 6 weeks at which point the pH was 8.39, a change of 1.28

Notably, all formulations were more stable when stored at lower temperatures, for example, when stored at about 5° C.

It is known that urea can decompose at alkaline pH to carbon dioxide and ammonia. (See, Swanbeck G. Urea in the treatment of dry skin. Acta Derm Venereol Suppl (Stockh). 1992 177:7-8.) However, no expansion of tubes was observed as the pH increased. This may be because the carbon dioxide formed bicarbonate within the vehicle or that the urea was not decomposing. It has also been known that adding lactic acid to a 10-20% urea solution prevents residue on the skin upon drying. It was thus proposed that lactic acid may be a good vehicle for urea not only in preventing residue, but also by lowering the pH of the formulations, Id. There was no reasoning, however, that there was any particular advantage of lactic acid over other pH lowering components. Moreover, so long as the urea formulation is prepared with a relatively low pH, for example less than 7.0, less than 6.2, or less than 6.0, the urea and the formulation would be expected to remain stable and active, and there was no reason to believe that pH would change or that the vehicle would have an impact on efficacy.

It was further found that increasing the amount of sodium lactate to 2.5% did not provide any further stability. It has also been proposed that the addition of gluconolactone to a urea formulation stabilizes the pH. However, addition of 2-8% gluconolactone to the urea formulation used to generate the data in Table 1 failed to provide sufficient stabilization to the formulation.

A new formulation was then prepared in an attempt to better stabilize the pH. Table 4 shows the formulation, with the last column showing the amount of ingredients in the comparative examples:

TABLE 4
Exemplary pH Stable Formulation.

| | | Weight Percent |
|---|---|---|
| | Weight Percent | in Comparative |
| Component | (% w/w) | Formulations |
| WHITE PETROLATUM, | 1.50 | 2.00 |
| USP | | |
| MINERAL OIL, USP | 3.50 | 4.00 |
| EMULSIFYING WAX, | 4.00 | 4.00 |
| NF | | |
| BHT | 0.10 | 0.10 |
| BENZOIC ACID | 0.25 | 0.25 |
| UREA, USP | 40.00 | 40.00 |
| PURIFIED WATER, USP | 39.70 | 38-41 |
| CARBOMER 980, NF | 1.50 | 1.5 |
| EDETATE DISODIUM, | 0.10 | 0.1 |
| USP | | |
| L-LACTIC ACID, USP | 1.00 | — |
| PROPYLENE GLYCOL, | 6.35 | 6.5 |
| USP | | |
| GLUCONOLACTONE, | 2.00 | — |
| USP | | |

A double-blind randomized study was conducted to evaluate two-week application of the pH stable urea cream vs. placebo (vehicle) for the treatment of plaque psoriasis in adult subjects. The study was a single-center study of a two-week application of the urea cream applied twice daily to the target areas of the skin. Patients were evaluated by means of photographs of lesions taken at the baseline visit compared to photographs of lesions after treatment. The modified formulation of urea in this second study was clinically superior to the formulation of urea in the first study. Furthermore, based on the scale portion of the Psoriasis Area Severity Index (sPASI) score, the urea cream showed a statistically significant improvement over vehicle. (Details given in the Examples presented below.)

The pH stability of the composition in Table 4 was also tested for pH stability under the same conditions as the Comparative examples (25° C., 60% RH). The initial pH was determined to be 5.00. After about eight weeks of storage, the pH was 5.28, a change of only 0.28. In contrast, the best performing comparative example showed a pH change of 1.12 after eight weeks of storage. This enhanced pH stability can account for the improved efficacy of the new composition as compared to both vehicle and the initial, comparative formulations. A second batch of the pH stable formulation was prepared with an initial pH of 5.20. After 13 weeks of storage under the same conditions, the pH had changed to 5.80, a change of 0.6 pH units. In contrast, comparative examples stored under the same conditions for a total of 9-11 weeks exhibited pH changes ranging from 1.42 to 1.87.

In accordance with the above, the present invention is a urea containing composition for the treatment of a skin disorder. The urea is present in a relatively high concentration, for example in amount of about 20% or greater, about 20% to about 40%, about 30% or greater, about 30% to about 40%, or about 40%.

Formulations according to the invention also include a pH stabilizing component. The pH stabilizing component includes a hydroxy acid and a polyhydroxy acid (or a lactone of the polyhydroxy acid). A hydroxy acid is a carboxylic acid that includes an additional hydroxy (—OH) group, i.e., a hydroxy group in addition to the —OH that forms part of the carboxyl (—COOH) group. Exemplary hydroxy acids include α-, β-, γ-, and δ-hydroxy acids. In some embodiments, the hydroxy acid is an α-hydroxy acid. In embodiments, the α-hydroxy acid is glycolic acid or lactic acid; in particular embodiments, the α-hydroxy acid is lactic acid. A polyhydroxy acid is a carboxylic acid that includes several additional hydroxy groups. As used herein, several means three or more, four or more or five or more. Exemplary polyhydroxy acids include gluconic acid and lactobionic acid. In some embodiments, the polyhydroxy acid is gluconic acid. The polyhydroxy acid may be in the form of a lactone; for example, gluconolactone is a lactone of gluconic acid. Gluconolactone is an exemplary polyhydroxy acid used in the pH stabilizing component.

The hydroxy acid is present in an amount of about 0.25% to about 15% in the final composition. The amount of hydroxy acid can vary depending on the desired initial pH. In exemplary embodiments with low to neutral pH, the hydroxy acid is present in an amount to obtain 0.25% to about 2% hydroxy acid in the final composition, about 0.5% to about 1.5% hydroxy acid in the final composition, or about 1.0% hydroxy acid in the final composition. Although the amounts described above refer to hydroxy acids in general, the quantities apply similarly whether the hydroxy acid is an α-hydroxy acid, β-hydroxy acid, γ-hydroxy acid, or a 6-hydroxy acid, or is glycolic acid or lactic acid.

The polyhydroxy acid or lactone thereof is present in an amount of about 0.5% to about 8% in the final composition, about 1% to about 3% polyhydroxy acid in the final composition, or about 2% polyhydroxy acid in the final composition. Although the amounts described above refer to polyhydroxy acids in general, the quantities apply similarly whether the hydroxy acid is linear such as gluconic acid, a lactone such as gluconolactone, or contains linear and lactone portions such as in lactobionic acid.

In an exemplary embodiment of the invention the pH stabilizing component includes a mixture of lactic acid (as a hydroxy acid component) and gluconolactone (as a polyhydroxy acid component). The amounts of lactic acid and gluconolactone are selected to achieve a desired initial pH. In embodiments, the desired pH is less than about 6.2, about 6.0 or less, about 5.5 or less, about 5.0 or less, or about 4.8 or less, when initially prepared. In any embodiment, the pH is above about 4.0, or above about 4.5, when initially prepared. In exemplary embodiments, the pH is from about 4.5 to about 6.5, from about 4.8 to about 6.2, from about 5.0 to about 6.0, or from about 4.8 to about 5.5, when initially prepared. In embodiments, the amount of lactic acid in the final composition can be from about 0.25% to about 2%, about 0.5% to about 1.5%, or about 1.0%. In some embodiments, the amount of lactic acid can be up to about 15%, particularly when a lower pH is desired. In embodiments, the amount of gluconolactone in the final composition can be from about 0.5% to about 5%, about 1% to about 3%, or about 2.0%. Some embodiments include about 40% urea, about 1.0% lactic acid, and about 2.0% gluconolactone.

Compositions according to the invention are pH stable. As used herein, pH stable means that there is no significant change in pH upon storage of the composition. For example, in some embodiments, compositions according to the invention exhibit a pH change of less than 0.5 units upon storage at 25° C. and 60% RH for six weeks. Embodiments of compositions according to the invention exhibit a pH change of less than 1.0 units upon storage at 25° C. and 60% RH for nine weeks or a pH change of less than 1.0 units upon storage at 25° C. and 60% RH for twelve weeks. Some embodiments of compositions according to the invention exhibit a pH change of less than 0.3 units upon storage at 25° C. and 60% RH for four weeks.

In embodiments, the composition is formulated to have an initial pH of less than 6.2. In such embodiments, the pH remains less than 6.2 after storage for four weeks at 25° C. and 60% RH, after storage for six weeks at 25° C. and 60% RH, after storage for eight weeks at 25° C. and 60% RH, or after storage for twelve weeks at 25° C. and 60% RH. For example, the pH at the time of manufacture can be about 5.0 to about 5.2 and, upon storage at 25° C. and 60% RH for 4 weeks or more, for example six weeks, eight weeks or twelve weeks, can remain less than about 6.2.

In some embodiments, the composition is formulated to have an initial pH of less than 6.0. In such embodiments, the pH remains less than 6.0 after storage for four weeks at 25° C. and 60% RH, after storage for six weeks at 25° C. and 60% RH, after storage for eight weeks at 25° C. and 60% RH, or after storage for twelve weeks at 25° C. and 60% RH. For example, the pH at the time of manufacture can be about 5.0 to about 5.2 and, upon storage at 25° C. and 60% RH for 4 weeks or more, for example six weeks, eight weeks or twelve weeks, can remain less than about 6.0.

In embodiments, the pH change upon storage at 25° C. and 60% RH can be about 0.3 pH units or less after six weeks, about 0.35 pH units or less after eight weeks, or about 0.6 pH units or less after twelve weeks.

Exemplary compositions according to the invention are prepared as an emulsion, which may take the form of a cream or a lotion. Exemplary compositions according to the invention are creams. Compositions are formed using methods generally known in the art by combining an aqueous phase with a non-aqueous hydrophobic phase. The aqueous phase contains water as a solvent and the non-aqueous phase contains an oil as a solvent. Mineral oil is an exemplary non-aqueous solvent, although others known in the art, for example silicone oil and natural oils. The non-aqueous phase may include other hydrophobic components such as petrolatum (or petroleum jelly), waxes and fatty acids.

An exemplary embodiment of the invention includes mineral oil, white petrolatum and emulsifying wax in the non-aqueous phase. For example, the non-aqueous phase may contain mineral oil in an amount to obtain about 3.0% to about 5.0% mineral oil in the final composition, about 3.0% to about 4.0% mineral oil in the final composition, or about 3.5% mineral oil in the final composition. The non-aqueous phase may contain white petrolatum in an amount to obtain about 1.0% to about 2.0% white petrolatum in the final composition or about 1.5% white petrolatum in the final composition. The non-aqueous phase may contain an emulsifying wax in an amount to obtain about 3.0% to about 5.0% emulsifying wax in the final composition or about 4.0% emulsifying wax in the final composition. Additional components may be added to the non-aqueous phase prior to combining with the aqueous phase. Such ingredients can include preservatives and stabilizers, for example butylated hydroxytoluene (BHT) and benzoic acid. Preservatives, stabilizers, and other components added to the non-aqueous phase are generally water insoluble components, or components more soluble in oil than in water.

The aqueous phase used in preparing compositions of the invention has water as the primary solvent, and includes the urea and the pH stabilizing components in amounts described elsewhere herein. The aqueous phase can include water in an amount to obtain 30% to about 45% water in the final composition, about 35% to about 40% water in the final composition, or about 39.5% water in the final composition. Components in addition to urea and the pH stabilizing components may be added to the aqueous phase prior to combining with the non-aqueous phase. Such ingredients can include, for example, emulsifying agents, stabilizers, humectants, hydrating agents, and the like. For example, the aqueous phase can include Carbomers and other polymer emulsifying agents, disodium edetate as a stabilizing agent, and propylene glycol as a humectant/hydrating agent or for its antifungal and antimicrobial properties. Components added to the aqueous phase are generally water-soluble components or components more soluble in water than in the hydrophobic solvent of the non-aqueous phase.

The pH stabilizing components used in the present formulation include a hydroxy acid and a polyhydroxy acid, which may be present as a lactone. The hydroxy acid is a carboxylic acid that includes an additional hydroxy group, i.e., a hydroxy group in addition to the —OH that forms part of the carboxyl (—COOH) group. Exemplary hydroxy acids include α-, β-, γ-, and δ-hydroxy acids. In some embodiments, the α-hydroxy acid is glycolic acid or lactic acid. In particular embodiments, the α-hydroxy acid is lactic acid. The polyhydroxy acid is a carboxylic acid that includes several additional hydroxy groups. As used herein, several means three or more, four or more or five or more. Exemplary polyhydroxy acids include gluconic acid and lactobionic acid. In some embodiments, the polyhydroxy acid is gluconic acid. The polyhydroxy acid may be in the form of a lactone; for example, gluconolactone is a lactone of gluconic acid. Gluconolactone is a particularly exemplary polyhydroxy acid component.

The aqueous phase can include the hydroxy acid in an amount to obtain about 0.25% to about 15% hydroxy acid in the final composition, about 0.25% to about 2% hydroxy acid in the final composition, about 0.5% to about 1.5% hydroxy acid in the final composition, or about 1.0% hydroxy acid in the final composition. Although the amounts described above refer to hydroxy acids in general, the quantities apply similarly whether the hydroxy acid is an α-hydroxy acid, β-hydroxy acid, γ-hydroxy acid, a δ-hydroxy acid, glycolic acid or lactic acid. The aqueous phase can include the polyhydroxy acid or lactone thereof in an amount to obtain 0.5% to about 8% polyhydroxy acid in the final composition, about 1% to about 3% polyhydroxy acid in the final composition, or about 2% polyhydroxy acid in the final composition. Although the amounts described above refer to polyhydroxy acids in general, the quantities apply similarly whether the hydroxy acid is linear such as gluconic acid, a lactone such as gluconolactone, or contains linear and lactone portions such as lactobionic acid.

According to the present invention, compositions described herein are useful for and can be used for treatment of skin and nail conditions or disorders, as well as lesions affecting the hair and scalp. Such disorders are frequently associated with cornification. Disorders with associated cornification include hyperkeratotic lesions and other hyperkeratotic conditions. Hyperkeratotic conditions (i.e., skin disorders characterized by hyperkeratosis) are associated with an excessively thick horny layer of the epidermis, leading to painful, itchy, or disfiguring conditions with the potential for complications. Hyperkeratotic conditions include, but are not limited to, dry and rough skin, dermatitis, psoriasis (including plaque psoriasis), verruca vulgaris (warts), lichen simplex chronicus (LSC) xerosis, ichthyosis, eczema, actinic keratosis, stucco keratosis, seborrheic keratosis, keratosis pilaris, keratosis palmaris or plantaris, keratoderma, corns, calluses, lichen planus, pityriasis rubra pilaris, dermatophyte infection and any skin condition associated with scales. Hyperkeratotic conditions can also involve the nails leading to potential discomfort, disfigurement, or infection. Disorders of the nails, including hyperkeratotic conditions, include, but are not limited to, onychomycosis, psoriatic nails, damaged nails, ingrown nails and devitalized nails.

In exemplary embodiments, the composition acts as a keratolytic agent, meaning that it separates or loosens the horny outer layer of the epidermis associated with cornification. The composition is also emollient, or soothing to the skin, and can help soften hyperkeratotic skin and nails. The composition is also useful to treat conditions with indications for enzymatic debridement and promotion of normal healing of surface lesions, particularly where healing is retarded by local infection; necrotic, or dead tissue; fibrinous debris: debris composed of fibrin, a protein involved in blood clotting; purulent debris: debris containing, consisting of, or forming pus; and eschar.

According to the invention, an effective amount of the composition is applied to the skin or nail. The cream is smoothed onto the skin until absorbed, avoiding surrounding tissue. Optionally, the treated area may be occluded. In embodiments, the cream is applied once daily or twice daily. Treatment can continue for one week, two weeks, or indefinitely, until the desired endpoint is reached. For use on nails, the cream can be applied and occluded as necessary to achieve the desired effect.

The following non-limiting examples further describe aspects of the invention. These are exemplary and modifications of the precise examples can be made in view of the skill in the art and the description provided above.

### Example 1—Exemplary Inventive Formulation

An exemplary formulation according to the invention includes the ingredients shown in Table 5, below.

TABLE 5
Exemplary Inventive Composition

| | Weight Percent |
|---|---|
| Material Description | (% w/w) |
| WHITE PETROLATUM, USP | 1.50% |
| MINERAL OIL, USP | 3.50% |
| EMULSIFYING WAX, NF | 4.00% |
| BUTYLATED | 0.10% |
| HYDROXYTOLUENE, NF | |
| BENZOIC ACID POWDER, USP | 0.25% |
| UREA, USP | 40.00% |
| PURIFIED WATER, USP | 39.70% |
| CARBOMER 980, NF | 1.50% |
| EDETATE DISODIUM, USP | 0.10% |
| L-LACTIC ACID, USP | 1.00% |
| PROPYLENE GLYCOL, USP | 6.35% |
| GLUCONOLACTONE, USP | 2.00% |

### Example 2—Initial Comparative Composition

The Initial Comparative Composition used in the clinical trial described above and in Example 6 below is shown in Table 6. The Initial Comparative Composition was not pH stable (See Example 5.)

TABLE 6
Initial Comparative Composition

| | Weight Percent |
|---|---|
| Material Description | (% w/w) |
| WHITE PETROLATUM | 2.00% |
| MINERAL OIL (DRAKEOL 35) | 4.00% |
| EMULSIFYING WAX | 4.00% |
| BUTYLATED | 0.10% |
| HYDROXYTOLUENE | |
| BENZOIC ACID | 0.25% |
| UREA | 40.00% |
| CARBOPOL 980, NF | 0.50% |
| EDETATE DISODIUM | 0.10% |
| SODIUM LACTATE | 1.50% |
| SOLUTION (50%) | |
| PROPYLENE GLYCOL | 6.65% |
| NaOH | 0.21% |
| PURIFIED WATER | 40.69% |

### Example 3—Additional Comparative Compositions

The components of Comparative Compositions A-F are presented in Tables 7-12. Comparative Compositions A-F were not pH stable (See Example 5.)

TABLE 7
Comparative Composition A

| | Weight Percent |
|---|---|
| Material Description | (% w/w) |
| WHITE PETROLATUM | 2% |
| MINERAL OIL | 4% |
| EMULSIFYING WAX | 4% |
| BHT | 0.1% |
| BENZOIC ACID | 0.25% |
| UREA | 40% |
| CARBOMER | 0.5% |
| DISODIUM EDETATE | 0.1% |
| PROPYLENE GLYCOL | 6.65% |
| NaOH SOLUTION (18%) | 1.15% |
| WATER | 41.25% |

TABLE 8
Comparative Composition B

| | Weight Percent |
|---|---|
| Material Description | (% w/w) |
| WHITE PETROLATUM | 2% |
| MINERAL OIL | 4% |
| EMULSIFYING WAX | 4% |
| BHT | 0.1% |
| BENZYL ALCOHOL | 0.25% |
| UREA | 40% |
| CARBOMER | 0.5% |
| DISODIUM EDETATE | 0.1% |
| PROPYLENE GLYCOL | 6.65% |
| TROMETHAMINE (40%) | 1.65% |
| WATER | 40.75% |

TABLE 9
Comparative Composition C

| | Weight Percent |
|---|---|
| Material Description | (% w/w) |
| WHITE PETROLATUM | 2% |
| MINERAL OIL | 4% |
| EMULSIFYING WAX | 4% |
| BHT | 0.1% |
| BENZOIC ACID | 0.25% |
| UREA | 40% |
| CARBOMER | 0.5% |
| DISODIUM EDETATE | 0.1% |
| SODIUM LACTATE | 0.75% |
| PROPYLENE GLYCOL | 6.65% |
| CITRIC ACID ANHYDROUS | 0.463% |
| SODIUM CITRATE | 2.233% |
| NaOH SOLUTION (18%) | 1.15% |
| WATER | 37.804% |

TABLE 10
Comparative Composition D

| | Weight Percent |
|---|---|
| Material Description | (% w/w) |
| WHITE PETROLATUM | 2% |
| MINERAL OIL | 4% |
| EMULSIFYING WAX | 4% |
| BHT | 0.1% |
| BENZYL ALCOHOL | 0.25% |
| UREA | 40% |
| CARBOMER | 0.5% |
| DISODIUM EDETATE | 0.1% |
| PROPYLENE GLYCOL | 6.65% |
| NaOH SOLUTION (18%) | 1.15% |
| WATER | 41.25% |

TABLE 11
Comparative Composition E

| | Weight Percent |
|---|---|
| Material Description | (% w/w) |
| WHITE PETROLATUM | 2% |
| MINERAL OIL | 4% |
| EMULSIFYING WAX | 4% |
| BHT | 0.1% |
| BENZOIC ACID | 0.25% |
| UREA | 40% |
| CARBOMER | 0.5% |
| DISODIUM EDETATE | 0.1% |
| SODIUM LACTATE | 0.75% |
| PROPYLENE GLYCOL | 6.65% |
| NaOH SOLUTION (18%) | 1.15% |
| WATER | 40.5% |

TABLE 12
Comparative Composition F

| | Weight Percent |
|---|---|
| Material Description | (% w/w) |
| WHITE PETROLATUM | 2% |
| MINERAL OIL | 4% |
| EMULSIFYING WAX | 4% |
| BHT | 0.1% |
| BENZOIC ACID | 0.25% |
| UREA | 40% |
| CARBOMER | 0.5% |
| DISODIUM EDETATE | 0.1% |
| PROPYLENE GLYCOL | 6.65% |
| CITRIC ACID ANHYDROUS | 0.463% |
| SODIUM CITRATE | 2.233% |
| TROMETHAMINE (40%) | 1.65% |
| WATER | 38.054% |

### Example 4—Stability of Inventive Formulation

A formulation according to Example 1 was prepared as a cream in the form of an oil in water emulsion. The formulation was packaged into aluminum laminate tubes (4 oz) and stored at 25° C. and 60% RH. The pH was measured according to USP 791. Briefly, a pH meter was calibrated using buffer solutions of known pH in ranges higher and lower than the initial pH of the test material. The pH probe was inserted directly into the cream and the pH measured. Tables 13 and 14 summarize data from two different experiments.

TABLE 13
pH Stability of Inventive Composition

| Time (Days) | Time (Weeks) | pH |
|---|---|---|
| 0 | 0.00 | 5.00 |
| 36 | 5.14 | 5.27 |
| 57 | 8.14 | 5.28 |
| 113 | 16.14 | 5.39 |

TABLE 14
pH Stability of Inventive Composition

| Time (Days) | Time (Weeks) | pH |
|---|---|---|
| 0 | 0 | 5.20 |
| 92 | 13 | 5.80 |

### Example 5—pH Stability of Comparative Compositions

Tables 15 through 20 provide data for the Comparative Examples shown in Tables 2C through 2H, respectively. In all cases, the composition was prepared as a cream that is an oil in water emulsion and stored in aluminum laminate tubes (4 oz) at the indicated temperature and at 60% RH. The pH was measured as above according to USP 791.

TABLE 15
pH Stability Data for Comparative Composition A

| | pH at Week: | | | | | | | | | | |
|---|---|---|---|---|---|---|---|---|---|---|---|
| | 0 | 1.5 | 4 | 4.5 | 5.5 | 6.5 | 7.5 | 8.5 | 9.5 | 10.5 | 11.5 |
| pH @ 5 C. | 6.80 | 6.93 | 6.93 | 7.01 | 6.92 | 6.92 | 6.94 | 6.45 | 7.02 | 6.93 | 7.08 |
| pH @ 25 C. | 6.80 | 7.02 | 7.48 | 7.63 | 7.64 | 7.87 | 7.9 | 8.13 | 8.2 | 8.46 | 8.67 |
| pH @ 30 C. | 6.80 | 7.25 | 7.82 | 7.96 | 8.03 | 8.27 | 8.15 | 8.52 | 8.52 | 8.68 | 8.98 |
| pH @ 40 C. | 6.80 | 8.20 | 9.09 | 9.17 | 9.13 | 9.27 | 9.07 | 9.33 | 9.17 | 9.2 | 9.59 |

TABLE 16
pH Stability Data for Comparative Composition B

| | pH at Week: | | | | | | | | | | |
|---|---|---|---|---|---|---|---|---|---|---|---|
| | 0.00 | 1 | 3.5 | 4 | 5 | 6 | 7 | 8 | 9 | 10 | 11 |
| pH @ 5 C. | 6.46 | 6.66 | 6.58 | 6.65 | 6.57 | 6.57 | 6.67 | 6.6 | 6.68 | 6.65 | 6.6 |
| pH @ 25 C. | 6.46 | 6.78 | 7.12 | 7.29 | 7.26 | 7.48 | 7.44 | 7.62 | 7.67 | 7.82 | 7.95 |
| pH @ 30 C. | 6.46 | 6.87 | 7.38 | 7.50 | 7.50 | 7.70 | 7.65 | 7.91 | 7.89 | 8.04 | 8.27 |
| pH @ 40 C. | 6.46 | 7.69 | 8.48 | 8.76 | 8.74 | 8.92 | 8.76 | 9.13 | 9.02 | 9.08 | 9.48 |

TABLE 17
pH Stability Data for Comparative Composition C

| | pH at Week: | | | | | | | | | |
|---|---|---|---|---|---|---|---|---|---|---|
| | 0.00 | 1 | 2 | 3 | 4 | 5 | 6 | 7 | 8 | 9 |
| pH @ 5 C. | 6.47 | 6.60 | 6.43 | 6.42 | 6.5 | 6.45 | 6.45 | 6.4 | 6.55 | 6.56 |
| pH @ 25 C. | 6.47 | 6.58 | 6.53 | 6.61 | 6.86 | 6.92 | 7 | 7.36 | 7.59 | 7.89 |
| pH @ 30 C. | 6.47 | 6.62 | 6.59 | 6.85 | 6.92 | 7.37 | 7.56 | 8.03 | 8.46 | 8.5 |
| pH @ 40 C. | 6.47 | 7.11 | 7.82 | 8.47 | 8.67 | 8.98 | 9.02 | 9.06 | 9.46 | 9.2 |
| pH @ 50 C. | 6.47 | 8.72 | 9.36 | 9.54 | 9.38 | 9.66 | 9.6 | 9.35 | | |

TABLE 18
pH Stability Data for Comparative Composition D

| | pH at Week: | | | | | | | | | | |
|---|---|---|---|---|---|---|---|---|---|---|---|
| | 0.00 | 1.5 | 4 | 4.5 | 5.5 | 6.5 | 7.5 | 8.5 | 9.5 | 10.5 | 11.5 |
| pH @ 5 C. | 7.29 | 7.40 | 7.43 | 7.49 | 7.38 | 7.38 | 7.44 | 7.41 | 7.44 | 7.51 | 7.54 |
| pH @ 25 C. | 7.29 | 7.53 | 7.95 | 8.10 | 8.16 | 8.40 | 8.44 | 8.66 | 8.71 | 8.91 | 9.1 |
| pH @ 30 C. | 7.29 | 7.71 | 8.40 | 8.55 | 8.64 | 8.85 | 8.73 | 9.01 | 9 | 9.05 | 9.36 |
| pH @ 40 C. | 7.29 | 8.68 | 9.34 | 9.43 | 9.36 | 9.46 | 9.23 | 9.49 | 9.38 | 9.3 | 9.72 |

TABLE 19
pH Stability Data for Comparative Composition E

| | pH at Week: | | | | | | | | | |
|---|---|---|---|---|---|---|---|---|---|---|
| | 0.00 | 0.5 | 1 | 1.5 | 2 | 2.5 | 3 | 4 | 5 | 6 |
| pH @ 5 C. | 7.11 | 7.27 | 7.18 | 7.30 | 7.35 | 7.43 | 7.48 | 7.39 | 7.65 | 7.61 |
| pH @ 25 C. | 7.11 | 7.36 | 7.45 | 7.74 | 7.72 | 8.11 | 8.21 | 8.45 | 8.59 | 8.39 |
| pH @ 30 C. | 7.11 | 7.38 | 7.57 | 7.94 | 8.09 | 8.37 | 8.46 | 8.61 | 8.86 | 8.8 |
| pH @ 40 C. | 7.11 | 8.00 | 8.62 | 8.91 | 8.87 | 9.16 | 9.12 | 9.16 | 9.46 | 9.29 |
| pH @ 50 C. | 7.11 | 8.93 | 9.39 | 9.58 | 9.46 | 9.62 | 9.52 | 9.27 | | |

TABLE 20
pH Stability Data for Comparative Composition F

| | pH at Week: | | | | | | | | |
|---|---|---|---|---|---|---|---|---|---|
| | 0.00 | 1 | 2 | 3 | 4 | 5 | 6 | 7 | 8 |
| pH @ 5 C. | 5.85 | | 6.06 | 5.99 | 6.04 | 6.18 | 5.98 | 6.09 | 6.2 |
| pH @ 25 C. | 5.85 | 6.01 | 6.07 | 6.25 | 6.36 | 6.67 | 6.8 | 6.93 | 7.11 |
| pH @ 30 C. | 5.85 | 6.05 | 6.12 | 6.47 | 6.61 | 6.93 | 7.06 | 7.45 | 7.64 |
| pH @ 40 C. | 5.85 | 6.81 | 7.32 | 7.93 | 8.08 | 8.38 | 8.85 | 8.56 | 8.79 |
| pH @ 50 C. | 5.85 | 8.86 | 8.95 | 9.36 | 9.18 | 9.01 | | | |

### Example 6—Comparative Compositions with Gluconolactone

Aqueous solutions containing 40% urea and 0% to 8% gluconolactone were prepared in duplicate and the pH monitored over 90 days. Results are shown in Table 21.

TABLE 21
pH of Urea/Gluconolactone Solutions

| Gluconolactone | | Day | Day | Day | Day | Day | Day |
|---|---|---|---|---|---|---|---|
| (%) | Sample | 0 | 10 | 20 | 30 | 60 | 90 |
| 0 | A | 7.0 | 8.8 | 9.1 | 9.3 | 9.5 | 9.5 |
| 0 | B | 7.1 | 8.8 | 9.1 | 9.3 | 9.5 | 9.5 |
| 2 | A | 4.0 | 6.0 | 7.3 | 8.0 | 8.6 | 8.8 |
| 2 | B | 4.0 | 6.1 | 7.4 | 8.0 | 8.6 | 8.8 |
| 4 | A | 3.8 | 4.4 | 6.2 | 7.6 | 8.1 | 8.4 |
| 4 | B | 3.8 | 4.4 | 6.2 | 7.6 | 8.1 | 8.5 |
| 8 | A | 3.7 | 3.9 | 4.3 | 4.9 | 7.5 | 7.9 |
| 8 | B | 3.6 | 3.9 | 4.3 | 4.9 | 7.6 | 8.0 |

By way of comparison, the pH of 2%, 4% and 8% gluconolactone solutions without urea were 2.6, 2.4, and 2.1, respectively, and did not change by more than one pH unit over the 90-day period.

### Example 7—Comparative Compositions with Gluconolactone

An aqueous solution containing 40% urea and 2.5% lactic acid was prepared in duplicate and the pH monitored over 90 days. Results are shown in Table 22.

TABLE 22
pH of Urea/Lactic Acid Solutions

| Sample | Day 0 | Day 10 | Day 20 | Day 30 | Day 60 | Day 90 |
|---|---|---|---|---|---|---|
| A | 9.0 | 8.9 | 9.2 | 9.3 | 9.5 | 9.6 |
| B | 8.8 | 8.9 | 9.2 | 9.3 | 9.5 | 9.6 |

By way of comparison, the pH of a 2.5% lactic acid solution without urea was 9.0 and increased to 10.0 after 90 days.

### Example 8—Clinical Data for Initial Comparative Composition

A formulation according to Example 2 was prepared as a cream in the form of an oil in water emulsion. A double-blind, parallel group study was conducted to evaluate the urea cream (40%) for the treatment of ichthyosis in adult subjects for one-month.

STUDY DESIGN: A single center, randomized, double-blind, parallel group, comparison design, single concentration study was conducted on 19, adult, human, male and non-pregnant female patients affected with ichthyosis.
SYNOPSIS: Patients that were enrolled in the study self-administered either the Test product (A) or Placebo (B) twice daily to the target area on both legs for a period of four weeks except for the first application of the study treatment, which was applied at the time of enrollment. The treatment administration was determined by a computer-generated randomization assignment. At the first visit, the investigator designated an area on each leg as the target area. Photographs of the target area were made at the initial visit and at each subsequent visit. Patients were given a Daily Personal Diary and containers of study medication, DOVE® unscented bar soap, together with instructions on how to wash the affected area and apply the medication. In addition to recording the time and date of each medication application, the Daily Personal Diaries were to be used to record all concomitant medication taken and all adverse events experienced. There were 9 patients assigned to the treatment group and 10 patients assigned to the placebo group.
Patients were evaluated at baseline, two weeks, four weeks and two weeks after the end of treatment, using an Ichthyosis Area and Severity Scale (IASS) developed by the sponsor. The IASS was developed as follows:

### Target Area Ichthyosis Severity Scale

### Scaling:

Size of plates:

0=No scaling

1=Small, <3 mm

2=Medium, 3-5 mm

3=Large, >5 mm

Hyperpigmentation of scales:

0=No difference from background skin

1=Central pigmentation

Lifting of edges of scales:

0=No lifting

1=Edges are lifted on at least 25% of the scales

2=More than the edges are lifted on at least 10% of the scales

### Fissuring:

Space between scales:

0=None

1=Thin (barely perceptible linear outlines)

2=Thick

Contiguity of spaces between scales:

0=Not connected

1=Somewhat connected, up to 80% connected linear outlines

2=Overwhelmingly connected, >80% connected linear outlines

### Methodology:

1) Score each of the following body areas by themselves:
head (H)=10% BSA
arms (A)=20% BSA
trunk (T)=30% BSA
legs (L)=40% BSA

2) Combine the four scores for the final IASI.
a) For each section, the percent of area of skin involved, is estimated and then transformed into a grade from 0 to 6 (BSA Grade):
0% of involved area, grade: 0
<10% of involved area, grade: 1
10-29% of involved area, grade: 2
30-49% of involved area, grade: 3
50-69% of involved area, grade: 4
70-89% of involved area, grade: 5
90-100% of involved area, grade: 6
b) Within each area, measure the severity of each of five clinical signs (using grades in parentheses from none to maximum as defined by the Target Area Ichthyosis Severity Scale above):
Size of plates (0-3)
Hyperpigmentation of scales (0-1)
Lifting of edges of scales (0-2)
Space between scales (0-2)
Contiguity of spaces between scales (0-2)
c) Calculate the sum of all five severity parameters for each section of skin, multiplied by the area score for that area and multiplied by the weight of the respective section (0.1 for head, 0.2 for arms, 0.3 for trunk and 0.4 for legs).
   Total score can range from 0 to 60.
   The table below gives the scores for a worst-case scenario of disease severity:

| Body Part | H | A | T | L |
|---|---|---|---|---|
| BSA Grade | 6 | 6 | 6 | 6 |
| Size of plates | 3 | 3 | 3 | 3 |
| Hyperpigmentation | 1 | 1 | 1 | 1 |
| Lifting | 2 | 2 | 2 | 2 |
| Space between | 2 | 2 | 2 | 2 |
| Contiguity | 2 | 2 | 2 | 2 |
| Sub score | 60 | 60 | 60 | 60 |
| Weighted area | 6 | 12 | 18 | 24 |

Total IASI: 60

### Results:

Urea Ichthyosis Trial

| | Active | Placebo | | |
|---|---|---|---|---|
| | Mean ichthyosis | Mean ichthyosis | | |
| | score | score | Difference | P value¹ |
| Visit 1 | 6.6 | 6.45 | 0.16 | 0.6700 |
| Visit 2 | 4.3 | 3 | 1.33 | 0.2292 |
| Visit 3 | 4.8 | 2.9 | 1.93 | 0.0519 |
| Visit 4 | 3.9 | 4.5 | −0.61 | 0.4341 |

¹Least-Squares Mean

There was no statistically significant difference in the clinical outcome when urea was compared to vehicle. In assessing the results, we observed that the pH of the placebo (vehicle alone) did not change while the pH of the urea rose from the time initially manufactured to the time tested. (Data presented in Table 1, above)

### Example 9—Clinical Data for Inventive Composition

A formulation according to Example 1 was prepared as a cream in the form of an oil in water emulsion. A double-blind randomized study was conducted to evaluate a two-week application of urea cream 40% or placebo for the treatment of plaque psoriasis in adult subjects. Results are expected to be similar to those observed with ichthyosis.
STUDY DESIGN: Single-center double-blind study of a two-week application of Urea Cream 40% or placebo applied twice daily to the target areas of the skin. Patients were evaluated by means of photographs of lesions taken at the baseline visit compared to photographs of lesions after treatment. A group of 40 patients with plaque psoriasis were studied for two weeks. Patients that were enrolled in the study self-administered either the Test product (A) or Placebo (B). The treatment administration was determined by a computer-generated randomization assignment. At the first visit, the investigator designated an area on each leg as the target area. Photographs of the target area were made at the initial visit and at each subsequent visit. Patients were given a Daily Personal Diary and containers of study medication, DOVE® unscented bar soap, together with instructions on how to wash the affected area and apply the medication. In addition to recording the time and date of each medication application, the Daily Personal Diaries were to be used to record all concomitant medication taken and all adverse events experienced. Urea Cream 40% or Placebo was applied twice daily to target areas of the skin for a period of two weeks. Patients were evaluated at baseline, and after two weeks. There were 20 patients assigned to the treatment group and 20 patients assigned to the placebo group.
RESULTS: Results of statistical analyses for change (reduction) from baseline in the scale portion of the target lesion sPASI (scale Psoriasis Area Severity Index) score at Day 15 are summarized in Table 23. Statistical analysis was conducted according to standard industry practice. (See, for example, Sanford Bolton, Charles Bon “Pharmaceutical Statistics: Practical and Clinical Applications” 5th Ed. (CRC Press, 2009)

TABLE 23
Change in PASI Scores at Day 15

| | | | | 95% Confidence | | | |
|---|---|---|---|---|---|---|---|
| | | | | Interval | | | |
| | Means | | | Lower | Upper | ANOVA | Wilcoxon* |
| Evaluator | Urea | Placebo | Difference | Limit | Limit | p-value | p-value |
| Pictures | 1.65 | 0.70 | 0.950 | 0.255 | 1.645 | 0.0087 | 0.0053 |

*Wilcoxon non-parametric test exact p-value.

Results of statistical analyses for final sPASI score at Day 15 are summarized in Table 24. Urea treatment resulted in a lower sPASI score than placebo after two weeks.

TABLE 24
Final sPASI Scores at Day 15

| | | | | 95% Confidence | | |
|---|---|---|---|---|---|---|
| | | | Placebo − | Interval | | |
| | LS Means | | Urea | Lower | Upper | ANCOVA |
| Evaluator | Urea | Placebo | Difference | Limit | Limit | p-value* |
| Pictures | 1.42 | 2.38 | 0.958 | 0.259 | 1.657 | 0.0085 |

*Analysis of Covariance using Baseline sPASI as the covariate; LS means are adjusted ones.
** Baseline p-value > 0.05 indicating it was not an influential covariate in the evaluation.

The urea formulation of Example 1 as tested was superior to placebo. It is noteworthy, that the utilization of photographs of the lesion on both entrance into the study (baseline) and at week 2, made the evaluation less dependent upon subjective assessment of an individual evaluator and allowed for assessment by multiple independent evaluators as needed.

Summary:

In the first study the pH of Comparative Urea Cream 40% and Vehicle Cream were about 9.4 and about 5, respectively. In the second study the pH of the inventive Urea Cream 40% and Vehicle Cream were 5-6 and about 7, respectively. Vehicle Cream was titrated to a neutral pH 7.2. In the second study, Urea Cream 40% was expected to be more effective, Vehicle Cream less effective. If pH were not a factor, then similar results would have been expected with both studies. As described above, the inventive formulation (Example 1) maintains a pH of less than about 6.2 or less than about 6.0 for an extended period of time. The clinical superiority of the inventive formulation shows that the benefits of the composition are dependent upon the pH. Thus, the ability of the inventive composition to maintain an acceptable pH of less than 6.2, or less than about 6.0 results in superior performance after storage.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A composition for the treatment of a skin disorder, comprising:
at least 20% w/w urea, and
a pH stabilizing component comprising
a hydroxy acid and
a polyhydroxy acid or lactone thereof,
wherein the composition has an initial pH of less than 6.2, and
the composition has a pH less than about 6.2 after storage for at least four weeks.

2. The composition of claim 1, wherein the hydroxy acid is present in an amount of from about 0.25% to about 15%.

3. The composition of claim 1, wherein the hydroxy acid is present in an amount of from about 0.25% to about 2%.

4. The composition of claim 1, wherein the polyhydroxy acid or lactone thereof is present in an amount of from about 0.5% to about 8%.

5. The composition of claim 1, wherein the hydroxy acid is lactic acid.

6. The composition of claim 1, wherein the polyhydroxy acid is gluconic acid.

7. The composition of claim 6, wherein the gluconic acid is in the form of gluconolactone.

8. The composition of claim 1, wherein the hydroxy acid is lactic acid and the polyhydroxy acid or lactone thereof is gluconolactone.

9. The composition of claim 1, wherein the pH is less than about 6.2 after 4 weeks of storage at 25° C. and 60% Relative Humidity (RH).

10. The composition of claim 10, wherein the initial pH is from about 4.8 to about 6.2.

11. The composition of claim 1, comprising from about 20% to about 40% w/w urea.

12. The composition of claim 1, comprising about 40% w/w urea.

13. The composition of claim 11, comprising about 0.25% to about 2% lactic acid as the hydroxy acid.

14. The composition of claim 11, comprising about 0.5% to about 8% gluconolactone as the polyhydroxy acid or lactone thereof.

15. The composition of claim 13, comprising about 1% w/w lactic acid as the hydroxy acid and about 2% w/w gluconolactone as the polyhydroxy acid or lactone thereof.

16. A method of treating a skin or nail disorder to a patient in need thereof, comprising topically administering to a subject with said disorder an effective amount of a composition according to claim 1.

17. The method of claim 16, wherein the skin disorder or nail disorder is selected from the group consisting of ichthyosis, psoriasis, onychomycosis, psoriatic nails, damaged nails, ingrown nails, devitalized nails, and a hyperkeratotic condition, and
wherein the. hyperkeratotic condition is selected from the group consisting of dry and rough skin, dermatitis, psoriasis (including plaque psoriasis), verruca vulgaris (warts), lichen simplex chronicus (LSC), xerosis, ichthyosis, eczema, keratosis, actinic keratosis, stucco keratosis, seborrheic keratosis, keratosis pilaris, keratosis palmaris et plantaris, keratoderma, corns, calluses lichen planus, pityriasis rubra pilaris, dermatophyte infection and any skin condition associated with scalesa hyperkeratotic condition.

18. A method of improving pH stability of a urea formulation having greater than about 20% w/w urea, comprising adding a stabilizing amount of a hydroxy acid and a polyhydroxy acid or lactone thereof.

19. The method of claim 18, wherein the formulation has an initial pH of less than about 6.2 and, after storage for four weeks, the pH is of less than about 6.2.

20. The method of claim 18, wherein the formulation comprises from about 20% w/w to about 40% w/w urea
